(19) United States
(12) Patent Application Publication
(10) Pub. No.: US 2026/0004886 A1
(43) Pub. Date: Jan. 1, 2026

(54) SYSTEM AND METHOD FOR MANAGEMENT OF COMPRESSED SEQUENCING FILES

(71) Applicant: Personalis, Inc., Fremont, CA (US)

(72) Inventor: Alexander Halley STRAM, Altadena, CA (US)

(21) Appl. No.: 19/251,587

(22) Filed: Jun. 26, 2025

Related U.S. Application Data
(60) Provisional application No. 63/665,079, filed on Jun. 27, 2024.

(51) Int. Cl. G16B 50/50; G16B 30/10

(52) U.S. Cl. CPC G16B 50/50; G16B 30/10

(57) ABSTRACT

Systems and methods for management of storing and analyzing genetic sequencing data. In some embodiments disclosed herein, a method for converting a compressed SAM file back into a raw FASTQ file, wherein the information of the raw FASTQ file is substantively identical to that which was stored in the original FASTQ file from which the compressed SAM file is based is provided. The method advantageously enables storage of the smaller compressed SAM files for reliable, efficient reconstruction of the original FASTQ file when needed.

### CROSS-REFERENCE TO RELATED APPLICATIONS

This application claims priority to U.S. Provisional Application No. 63/665,079, which was filed Jun. 27, 2024. The disclosure of the patent application is herein incorporated by reference in its entirety and for all purposes.

### FIELD

The present application generally relates to systems and methods for storing genetic sequencing data and, more specifically, but not exclusively, for management of compressed filetypes storing biological sequences.

### BACKGROUND

Next generation sequencing data presents a number of challenges with respect to storing and analyzing sequencing data, primarily due to the size of such datasets. For example, for products that can provide an advanced, personalized, tumor-informed liquid biopsy assay to detect molecular residual disease and cancer recurrence—such as NeXT Personal® from Personalis in Fremont, CA—approximately 2.3 billion sequencing reads are used in order to gain insights about a patient's tumor, enabling a custom assay to quantify minimal residual disease as the patient undergoes treatment. Altogether, the uncompressed “raw” sequencing data used for the initial bioinformatics analysis of a single patient represents nearly 850 gigabytes of information.

Of particular importance is the need to efficiently store sequencing data in a manner that enables rapid generation and reconstruction of data in various formats on the fly. Unfortunately, conventional solutions cannot provide this solution.

For example, FASTQ format is a text-based format for storing both a biological sequence (usually nucleotide sequence) and its corresponding quality scores. Both the sequence letter and quality score are each encoded with a single ASCII character for brevity. The FASTQ file format is the industry standard for storing un-aligned sequencing data, i.e., the “raw” sequencing data which represents base calls and associated quality scores as determined by the DNA/RNA sequencer and its associated analysis software. FASTQ files are typically used as the starting point of any sequencing data bioinformatics analysis workflow.

But FASTQ files typically contain up to millions of entries and can be several megabytes or gigabytes in size, which can make them too large to open and analyze by conventional text editors. Generally, conventional systems only use FASTQ files as input for tools that perform downstream analysis, such as alignment to a reference assembly.

The Sequence Alignment Map (SAM) file format is the industry standard for storing reference-based aligned sequence data in a text-based format. SAM files may be encoded in plaintext (SAM) or in a binary version of SAM (i.e., a Binary Alignment Map (BAM)), which uses block-level compression to compress sequence data with respect to the similarity/difference of the associated reference genome (i.e., Compressed Reference-Oriented Alignment Map (CRAM)). As used herein, for ease of discussion only, both BAM and CRAM encodings will be referred to as “compressed SAM” files.

It is often necessary to repeat bioinformatics analysis, starting from either un-aligned (FASTQ-formatted) or aligned (SAM-formatted) sequencing data. But due to the sheer size of files and the amount of computation necessary to repeat alignment, it is ideal to only store sequencing data in aligned SAM format, and regenerate FASTQ files if/when necessary. But conventional systems may not guarantee regeneration of FASTQ files that are substantively identical to the original FASTQ file.

In view of the foregoing, a need exists for an improved file management system and method for converting compressed SAM files back into raw FASTQ files in an effort to overcome the aforementioned obstacles and deficiencies of conventional file management systems.

### BRIEF DESCRIPTION OF THE DRAWINGS

The novel features of the disclosed systems and methods are set forth with particularity in the appended claims. A better understanding of the features and advantages of the present systems and methods will be obtained by reference to the following detailed description that sets forth illustrative embodiments, in which the principles of the invention are used, and the accompanying drawings (also “Fig.”, “FIG.”, “Figure”, “Figures”, “Figs.”, and “FIGs.” herein) of which:

FIG. 1 shows a top level block diagram illustrating one embodiment of data flow for managing a compressed sequencing file.

FIG. 2 shows a flow diagram illustrating one embodiment of the preprocessing steps of an input FASTQ file and a compressed SAM file of FIG. 1.

FIG. 3 shows a flow diagram illustrating one embodiment of the regeneration of a FASTQ file from a compressed SAM file of FIG. 1.

FIG. 4 shows an exemplary screenshot illustrating one embodiment of a BQSR model.

FIG. 5 shows an example computer, according to various embodiments.

It should be noted that the figures are not drawn to scale and that elements of similar structures or functions are generally represented by like reference numerals for illustrative purposes throughout the figures. It also should be noted that the figures are only intended to facilitate the description of the preferred embodiments. The figures do not illustrate every aspect of the described embodiments and do not limit the scope of the present disclosure.

### DETAILED DESCRIPTION

The description is presented to enable one of ordinary skill in the art to make and use the systems and methods and is provided in the context of a patent application and its requirements. The section headings used herein are for organization purposes only and are not to be construed as limiting the subject matter described. While various embodiments of the systems and methods of the present disclosure have been shown and described herein, it will be obvious to those skilled in the art that such embodiments are provided by way of example only. Numerous variations, changes, and substitutions may occur to those skilled in the art without departing from the invention(s). It should be understood that various alternatives to the embodiments of the systems and methods described herein may be employed in practicing any one of the systems and methods set forth herein.

All patents, published patent applications, other publications, and sequences from GenBank, and other databases referred to herein are incorporated by reference in their entirety with respect to the related technology.

### I. Definitions

Unless defined otherwise, technical, and scientific terms used herein have the same meaning as commonly understood by one of ordinary skill in the art to which the present disclosure belongs. For purposes of the present disclosure, the following terms are defined below. The definitions provided are intended to apply to a given term, as well as other derivative linguistic re-phrasings and grammatical equivalents of the term.

As used herein, the term “compressed SAM” refers to both Binary Alignment Map (BAM) files and Compressed Reference-Oriented Alignment Map (CRAM) files.

As used herein, the singular forms “a,” “an”, and “the” include plural referents unless the context clearly dictates otherwise. Thus, for example, reference to “an antigen” includes mixtures of antigens; reference to “a pharmaceutically acceptable carrier” includes mixtures of two or more such carriers, and the like. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A (alone)”, and “B (alone)”.

As used herein, the term “about” a value (or parameter) refers to ±10% of a stated value. When referring to a range of values (or parameters), the term “about” refers to +10% of the upper limit and −10% of the lower limit of a stated range of values. When a range of values is provided, it is to be understood that each intervening value between the upper and lower limit of that range, and any other stated or intervening value in that stated range, is encompassed within the scope of the present disclosure. Where the stated range includes upper and/or lower limits, ranges excluding either of those included limits are also included in the present disclosure.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Overview

The present disclosure is directed to systems and methods for management of storing and analyzing genetic sequencing data. In some embodiments disclosed herein, a method for converting a compressed SAM file back into a raw FASTQ file, wherein the information of the raw FASTQ file is substantively identical to that which was stored in the original FASTQ file from which the compressed SAM file is based is provided. The method advantageously enables storage of the smaller compressed SAM files for reliable, efficient reconstruction of the original FASTQ file when needed.

For example, in a typical workflow, sequencers can use various technology (e.g., cluster generation and sequencing by synthesis) to sequence millions or billions of clusters on a flow cell. For each cluster, base calls are made and stored for every cycle of sequencing by real-time-analysis software. When sequencing completes, the base calls are converted to sequence data, typically stored in a FASTQ file. The FASTQ file is a test file that contains the sequence data from the clusters that pass filters on a flow cell.

In some embodiments, each entry of the FASTQ file includes at least four line-separated fields per sequence: (1) sequence identifier; (2) sequence; (3) quality score identifier line; and (4) a quality score. Field 1 begins with a ‘@’ character and is followed by a sequence identifier and an optional description (like a FASTA title line). Field 2 is the raw sequence letters. Field 3 begins with a ‘+’ character and is optionally followed by the same sequence identifier (and any description) again. Field 4 encodes the quality values for the sequence in Field 2, and includes the same number of symbols as letters in the sequence.

The first field “Field 1” can be stored in two places: (1) The required SAM field QNAME stores the FASTQ sequence identifier; or (2) The optional description/comments is stored in the SAM format with a custom SAM tag.

The second field, “Field 2” can be stored in the SAM “SEQ” field.

The third field is typically assumed to be a “+”. The fourth field can either stored in the SAM “QUAL” field, or if these scores have been recalibrated via BQSR, a custom SAM tag is used to preserve the original contents of the fourth field. Additionally and/or alternatively, the BQSR model can be saved, and an inversion of the BQSR model is run on the SAM QUAL field. This can potentially save more space but introduce error.

Additional information regarding FASTQ files, for example, can be found in the bcl2fastq Conversion User Guide, available at https://support.illumina.com/content/dam/illumina-support/documents/documentation/software_documentation/bcl2fastq/bcl2fastq_letterbooklet_15038058brpmi.pdf, the FASTQ format Wikipedia, available at https://en.wikipedia.org/wiki/FASTQ_format, and the Sequence Alignment/Map Format Specification, available at https://samtools.github.io/hts-specs/SAMv1.pdf, which articles are hereby incorporated by reference in their entirety for all purposes. This original FASTQ file is often large.

Alignment data for large numbers of aligned reads are often output as sequence alignment and map (SAM) or binary alignment and map (BAM) files. An aligner usually takes in raw sequence data in the form of a FASTQ file along with a reference genome to generate a new file containing the reads as well as the genomic location from which they originated. But due to the sheer size of files and the amount of computation necessary to repeat alignment, it is ideal to only store sequencing data in aligned SAM format, and regenerate FASTQ files if/when necessary.

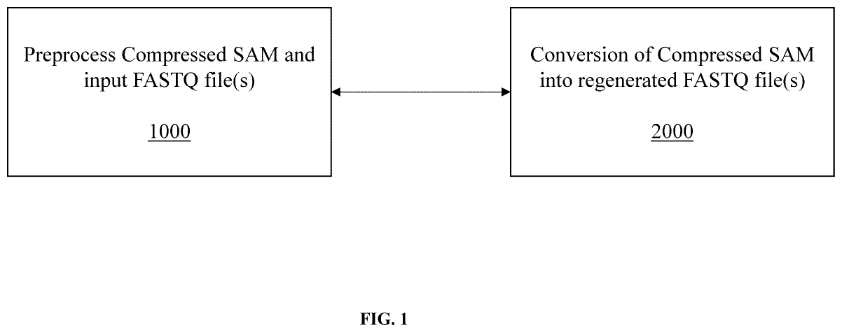

In some embodiments, a method for converting a compressed SAM file back into a raw FASTQ file is shown in FIG. 1. The disclosed methods are applicable to plaintext SAM, BAM, and CRAM encodings, and particularly useful for compressed SAM files. By providing a reliable method to recreate/regenerate a FASTQ file and verify its integrity, it is not necessary to store the original input FASTQ file. Instead, with the disclosed systems and methods, storing compressed SAM files are sufficient for preserving necessary sequencing data. As shown in FIG. 1, the method comprises at least two major subprocesses. Prior to any regeneration of a FASTQ file from a compressed SAM file, the method includes a preprocessing step 1000 of the compressed SAM file and the input FASTQ file on which the compressed SAM file is based. FIG. 1 also shows the process 2000 for regenerating a file that is substantively identical to the input FASTQ file based on the compressed SAM file created therefrom.

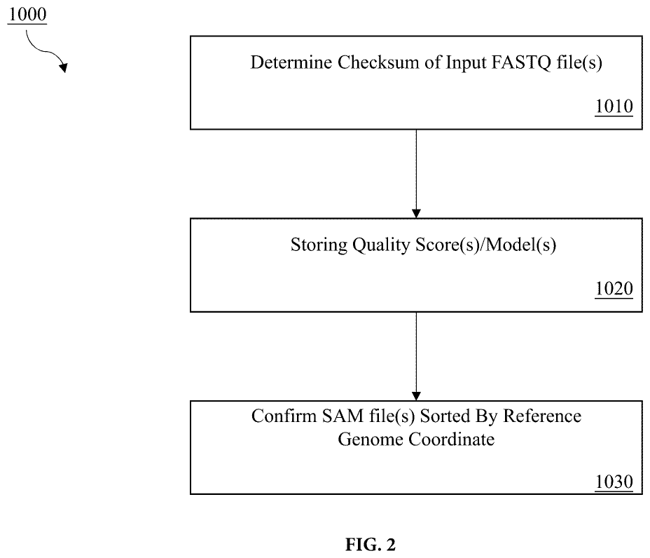

With reference to FIG. 2, the preprocessing step 1000 of the compressed SAM file and the input FASTQ file is shown in further detail. For example, once a compressed SAM file is generated from an input FASTQ file, the preprocessing step 1000 first determines a checksum—hereinafter referred to as FQSUM—of the input FASTQ file (process 1010), which is used for data integrity.

In some embodiments, when operating with tens of billions of base pairs of sequencing data, it is advantageous to remove redundant data wherever possible, primarily for the sake of resource/cost savings. For example, when operating at a scale where a computer's resources (e.g., memory/disk space) is easily overwhelmed, it can be advantageous to remove the original input FASTQ file once all information is stored in a CRAM file (which in fact has more information (alignment information), while using less disk space than the FASTQ). In other words, a file size of the CRAM file is much smaller than the original input FASTQ file it is based on. Therefore, the FQSUM can be calculated at any time before the original input FASTQ file is deleted.

The FQSUM is determined using an idempotent, commutative, and associative checksum algorithm. Stated in another way, for some set of one or more FASTQ records-A and B:

$$fqsum ( \{ A \} ) = fqsum ( fqsum ( \{ A \} ) )$$

$$and$$

$$fqsum ( \{ A , B \} ) = fqsum ( \{ B , A \} )$$

$$and$$

$$fqsum ( \{ fqsum ( \{ A , B \} ) , C \} ) = fqsum ( \{ A , fqsum ( \{ B , C \} ) \} )$$

Advantageously, the FQSUM can be used to verify data integrity without the need for sorting or re-ordering either the original input FASTQ files (or any intermediate output of the disclosed process). Compared to a conventional checksum, the FQSUM is advantageously order invariant. Since SAM files are typically stored in order of alignment (which reduces entropy and increases compression), converting directly from a SAM file to FASTQ, then running a checksum such as MD5 generally yields a different checksum than calculating the MD5 checksum on the original FASTQ input. Re-ordering the FASTQ output to resemble the input of the original FASTQ input is computationally prohibitive, as any such algorithm either requires: (1) a large amount of memory to run, generally equal to the amount of FASTQ data, or (2) require many iterations over the data and be very computationally slow. Since the order of the original FASTQ input is generally not meaningful (for example, pseudo-random), the FQSUM checksum advantageously ignores order and calculates the checksum on the set of reads (two sets being identical even if their elements appear in a different order). In some embodiments, the following pseudocode is used to determine the FQSUM:

```
fqsum_t fqsum(fq_t fastq_records) {
  fqsum_t fqsum_hash;
  for (record in fastq_records) {
      uint n = strlen(record.HEADER);
    for (uint i=0;i<n;i++)
        fqsum_hash−>HEADER[i] = (fqsum_hash−>HEADER[i] + record.HEADER[i]−32)%95;
    }
    assert(strlen(record.SEQ) == strlen(record.QUAL));
    n = strlen(record.SEQ);
    for (uint i=0; i<n;i++) {
        fqsum_hash−>SEQ[i] = (fqsum_hash−>SEQ[i] + record.SEQ[i]−33)%94;
        fqsum_hash−>QUAL[i] = (fqsum_hash−>QUAL[i] + record.QUAL[i]−33)%94;
    }
  }
  return fqsum_hash;
}
```

As shown in the pseudocode, the method to determine the FQSUM is invariant to the order of FASTQ records due to its commutative property. In other words, the FQSUM for the same set of FASTQ reads, either in a pseudo-random order output by sequencer software or in “sorted by alignment” order, advantageously are identical. Using the FQSUM for data integrity or verification is linear in time complexity and does not require sorting FASTQ files, which is typically impractical given the file sizes typically associated with conventional FASTQ files.

Turning back to FIG. 2, the preprocessing step 1000 of the compressed SAM file and the input FASTQ file then stores only the information necessary to construct original quality scores, even after the application of base quality score recalibration (BQSR) (process 1020). For example, a quality score can be a string of integers, equal in length to the sequence string, articulating the respective quality of each base call as it appears in the sequence string. If there are 100 bases in a read, there are 100 associated quality scores. Quality scores are generally represented on an integer scale of 0-91 (e.g., a Phred quality score). +33 is added and represented as ASCII characters with decimal representation 33 through 126, i.e., “!” is used to represent “0”, and “˜” is used to represent “91”.

Base quality scores are typically done at sequencing imaging time—a relative confidence of each base call is made, with Phred scaled score 0-91. Once a sequencing run is completed, the base quality scores is typically “recalibrated” by accounting for the entire dataset of quality scores, to ensure they make sense. BQSR is therefore a process where machine learning is applied to model these errors empirically and adjust the quality scores accordingly. For example, for a given run, whenever two A nucleotides are called in a row, the next base called had a 1% higher rate of error. Thus, any base call that comes after AA in a read should have its quality score reduced by 1%. That is repeated over several different covariates (mainly sequence context and position in read, or cycle) in a way that is additive. The same base may have its quality score increased for one reason and decreased for another.

In some embodiments, the recalibrated base quality scores appear in the same format as the original quality scores—e.g., a Phred scaled score (0-91)+33 and represented as an ASCII character. In other words, it is difficult to distinguish between “original” and “recalibrated” quality scores just by looking at the string of scores. In some embodiments, this storage is done by either storing the quality score model or by directly storing the original quality scores in a reserved SAM “tag.”

The BQSR model can be stored as a separate file, or within the SAM file “header”, for example, using an optional tag (e.g., @CO tag). For directly storing the original quality scores in a reserved SAM tag, the original quality string can be stored using an optional alignment field (e.g., @XQ tag). An example of storing the original quality score directly includes: XQ:Z:,:,FFF,,FFFFF:F:FFF,:FFF:,FFFFF:FFFFFFFF:FFFFF:FF::FF:FF:F,FF,,F:F,FF,FF,:F:FFF F:F:F:F:F:F,FF,FF:F:F,F,FFFFF:FFFFFFFFFFF:FFFFFFFFFFFFFFFFFFFFFFFFFFFFF

Where “XQ” is the tag name, and “Z” indicates the data is a string.

The actual quality string is: ,:,FFF,,FFFFF:F:FFF,:FFF:,FFFFF:FFFFFFFF:FFFFF:FF::FF:FF:F,FF,,F:F,FF,FF,:F:FFFF:F:F: F:F:F,FF,FF:F:F,F,FFFFF:FFFFFFFFFFF:FFFFFFFFFFFFFFFFFFFFFFFFFFFFF

Finally, at step 1030, the preprocessing step 1000 ensures that SAM records in the compressed SAM files are stored in a sorted order in order to reduce entropy and enable better compression of the compressed SAM file, as well as when compressing the FASTQ files generated by the present disclosure. In some embodiments, the SAM file can be sorted in a variety of methods to benefit the compression ratio. By way of example, two exemplary methods to sort the SAM file to ensure that entropy is reduced includes: (1) Sorting by reference genome coordinate(s); or (2) Sorting by sequence string.

In the context of the SAM specification, most bioinformatics tools expect a SAM file to be provided in sorted-by-reference-genome-coordinate order (method 1), and leverages the indexing mechanism available in SAM specification, which allows for quick retrieval of reads that fall within a provided reference genome coordinate.

In other embodiments, the SAM file is sorted by sequence string. Although less typical, similar reads, e.g., all reads that start with “AAA . . . ” appear together in the file. But SAM does not provide any indexing mechanism for this and retrieve reads are slower if the file is stored in this manner.

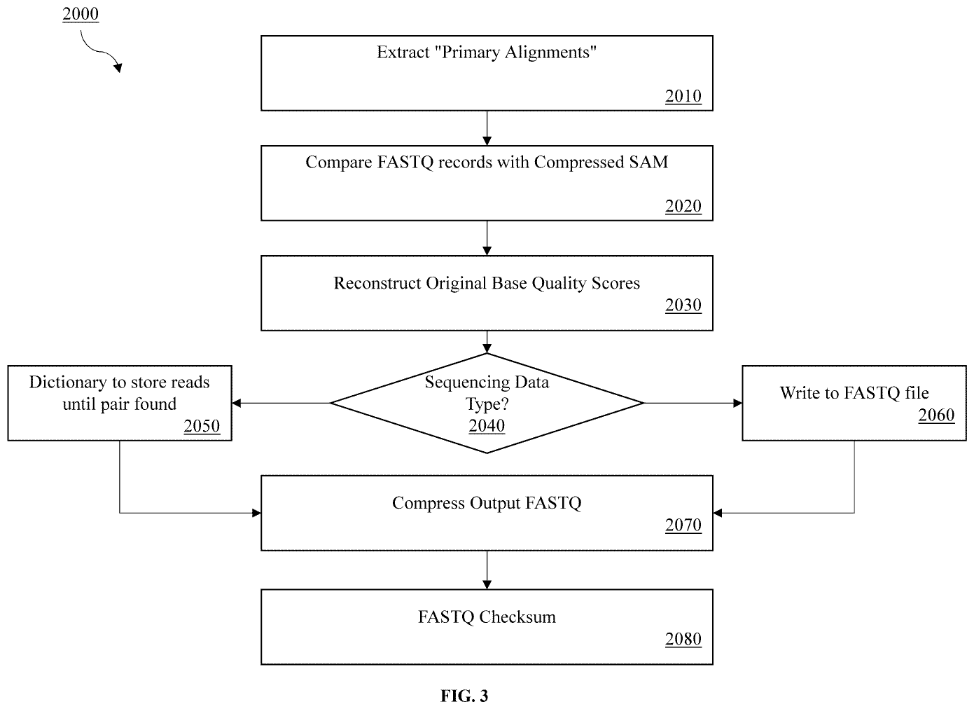

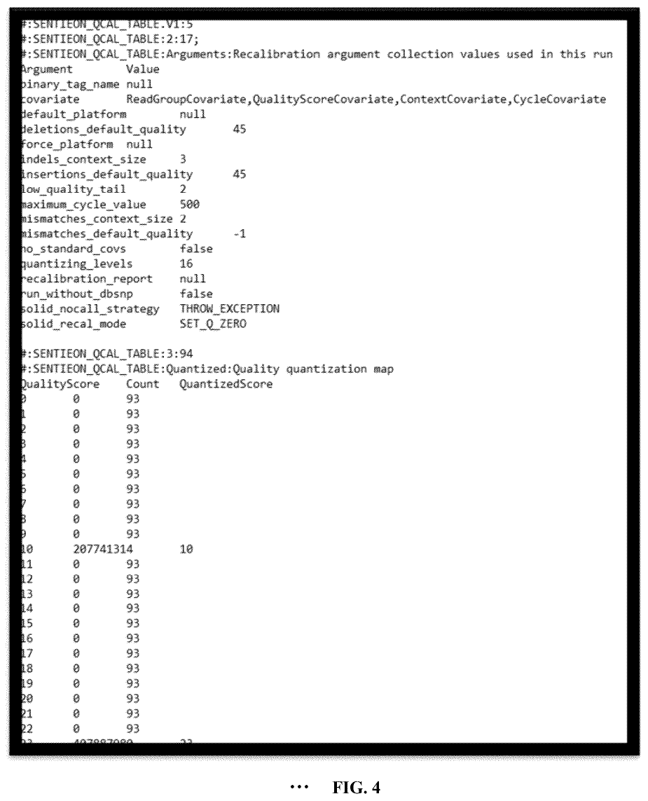

Following the preprocessing steps 1000, the process 2000 for regenerating a file that is substantively identical to the input FASTQ file based on the compressed SAM file created therefrom is detailed in FIG. 3. With reference to FIG. 3, the process 2000 begins by extracting only “primary alignments” from the compressed SAM file to ensure that there is no duplication of records in the output FASTQ files.

By way of example, assume a sequencing read with ID “ABC”. At the FASTQ level, the sequencing read IDs are unique with respect to each read or read-pair. If ABC only plausibly aligns to one part of the reference genome, then it will appear only once in the SAM file, and will be denotated as the “primary” alignment. There will be no secondary/supplementary alignment records for read “ABC”.

If ABC potentially aligns to different parts of the reference genome, multiple instances of sequence read “ABC” may be present in the SAM file. This is because the aligner indicates “it could map to location X, or Y or Z”. However, only one of these alignments is flagged to be “primary”. The other alignments are denoted as secondary or supplementary.

Since “ABC” should not appear multiple times in the reconstructed FASTQ file (as it only appeared once in the input), the system only considers the primary alignment.

At step 2020, the system confirms whether all FASTQ records of the original input FASTQ file is stored in the compressed SAM file (via the FQSUM). In some embodiments, it may be advantageous to remove sequencing duplicates or off-target reads and store these sequencing duplicates and/or off-target reads in an “auxiliary” compressed SAM file. For example, off-target reads include sequencing reads that are not directed to the targeted portion of the sequencing assay. During exome sequencing, to target the subset of DNA encoding proteins, primers and/or capture probes are configured with at least a subset designed to capture the flanking regions of a coding region. Frequently, there is sequencing beyond the coding portion, resulting in at least a portion of the read being off target. Sequence reads are usually generated over the course of the sequencing reaction and are subsequently reviewed/processed once the experiment is complete. If it is desired to exclude certain reads from the primary SAM file that is used for bioinformatics analysis, such as sequencing duplicates and/or off-target reads, the system can store such reads in the “auxiliary” compressed SAM file to ensure that such reads can still be reconstructed in FASTQ format at a later time in the case that bioinformatics analysis on these reads is later desired. In some embodiments, the “auxiliary” SAM file is identical in structure and format to the primary SAM file.

Furthermore, due to the properties of FQSUM, the system can then determine if FQSUM(original_FASTQ)=FQSUM(FQSUM(CHIEF_SAM_FASTQ), FQSUM(AUX_SAM_FASTQ)) to segregate “undesirable” reads from the initial bioinformatics analysis on the input SAM file, while still maintaining the flexibility to reconstruct those reads down the road if desired.

At step 2030, the original base quality scores are reconstructed either by grabbing directly from the reserved SAM tag, or applying the inverse of the BQSR model.

The BQSR model can be calculated on a per-sample basis, and is generally stored as a text file, used as a “ruleset” for the BQSR process to recalibrate reads.

If recalibrated_qualities=bqsr (model, original_qualities), this process can be inverted by saving the BQSR model:

original_qualities=bqsr⁻¹(model,recalibrated_qualities)

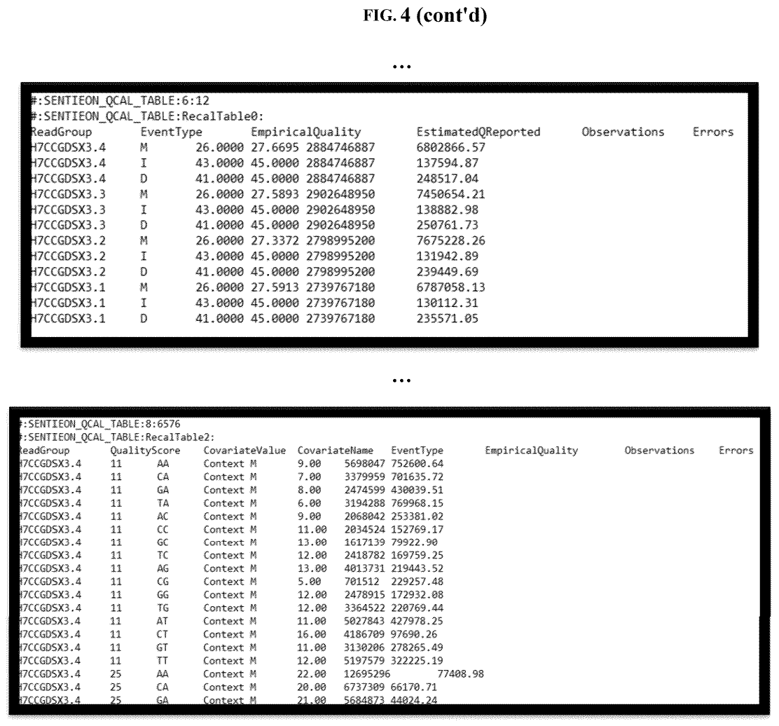

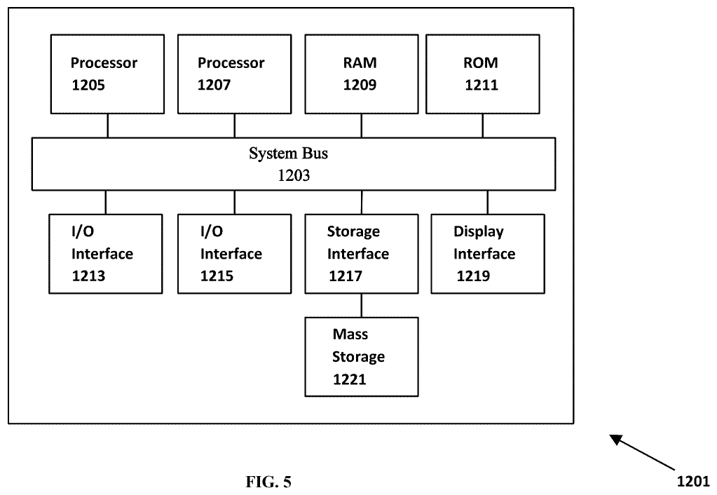

An exemplary screenshot of an exemplary BQSR file is shown in FIG. 4.

If using paired-end sequencing data (decision 2040), a dictionary is used to store reads in memory until its “mate” is found (step 2050). In some embodiments, paired-end sequencing data is reflected in the presentation of data in FASTQ format. Typically, paired-end sequencing data where two reads are “mated” together and have the same sequence identifier is received via pairs of FASTQ files. For example, “read 1” of each read pair is in a “read 1” FASTQ file, and “read 2” of each read pair is in a “read 2” FASTQ file. Both reads of a read pair can be stored in the same SAM file—there is a “SAM flag” which indicates whether the read is read 1 or read 2.

The disclosed method parses through the SAM file. If read 1 appears first, the system stores read 1 in memory until read 2 is found, and vice versa. When both “read 1” and “read 2” of a certain sequence identifier are available, read 1 will be written to the “read 1” FASTQ file, and “read 2” will be written to the “read 2” FASTQ file.

Paired FASTQ files must have reads appear in the same order. If read with sequence ID “ABC” appears as the 3rd record in the “Read 1” FASTQ file, it must also be the 3rd record in the “Read 2” FASTQ file. The disclosed method ensures this happens seamlessly.

Once a read and its mate are available, the memory should be freed and each read written to its corresponding FASTQ file. This will ensure that the output FASTQ files are roughly “sorted by alignment” and will compress better than the original “pseudo-random order” FASTQ file from the sequencing software. In other words, the regenerated FASTQ files output by the systems and methods disclosed herein are smaller than those typically generated by the original input FASTQ file generated by DNA sequencing software. It is noted that although the order of reads from the sequencer of the original FASTQ file is “pseudo-random,” this is typically a result of the physical coordinates of the molecule being sequenced with respect to the flowcell. For example, all reads from a specific tile are grouped together. This can be seen when looking at the FASTQ “header” record, which includes the tile number and the X-Y coordinates of the cluster within the tile. The actual “SEQ” and “QUAL” columns of the FASTQ record (ignoring the header) suggest the order does appear to be random, as this reflects the random process of molecules falling on different physical coordinates of the flowcell. Therefore, although the reads is not actually random, as used herein, the order appears pseudo-random.

Instead, at decision 2040, if using single-ended sequencing data, necessary information (e.g., three or four fields of the FASTQ format) is written to the FASTQ file as it is encountered in the compressed SAM file, which is sorted by alignment (step 2060). This will ensure that the output FASTQ file will be “sorted by alignment” and will compress better than the original “pseudo-random order” FASTQ file from the sequencing software.

The resultant FASTQ file (from either step 2050/2060) is compressed (at step 2070). This compressed FASTQ file is smaller than the original input FASTQ file due to the decreased entropy of FASTQ records that have been re-ordered to “sorted by alignment”, allowing for similar/overlapping reads to fall in the same compression window.

Finally, at step 2080, the FQSUM checksum is used, which, as discussed, is invariant to the order of FASTQ records to ensure that the FQSUM checksum of the FASTQ files output is identical to the original input FASTQ files that were originally created by sequencing software, which FQSUM was also stored in the SAM file header at step 1010. This checksum process does not require any pre-sorting or re-ordering of FASTQ records, making it ideal for large data sets.

### Hardware and Software

According to various embodiments, various functionality discussed herein can be performed by and/or with the help of one or more computers. Such a computer can be and/or incorporate, as just some examples, a personal computer, a server, a smartphone, a system-on-a-chip, and/or a microcontroller. Such a computer can, in various embodiments, run Linux, MacOS, Windows, or another operating system.

Such a computer can also be and/or incorporate one or more processors operatively connected to one or more memory or storage units, wherein the memory or storage may contain data, algorithms, and/or program code, and the processor or processors may execute the program code and/or manipulate the program code, data, and/or algorithms. Shown in FIG. 12 is an example computer employable in various embodiments of the present invention. Exemplary computer 1201 includes system bus 1203 which operatively connects two processors 1205 and 1207, random access memory (RAM) 1209, read-only memory (ROM) 1211, input output (I/O) interfaces 1213 and 1215, storage interface 1217, and display interface 1219. Storage interface 1217 in turn connects to mass storage 1221. Each of I/O interfaces 1213 and 1215 can, as just some examples, be a Universal Serial Bus (USB), a Thunderbolt, an Ethernet, a Bluetooth, a Long-Term Evolution (LTE), a 5G, an IEEE 488, and/or other interface. Mass storage 1221 can be a flash drive, a hard drive, an optical drive, or a memory chip, as just some possibilities. Processors 1205 and 1207 can each be, as just some examples, a commonly known processor such as an ARM-based or x86-based processor. Computer 1201 can, in various embodiments, include or be connected to a touch screen, a mouse, and/or a keyboard. Computer 1201 can additionally include or be attached to card readers, DVD drives, floppy disk drives, hard drives, memory cards, ROM, and/or the like whereby media containing program code (e.g., for performing various operations and/or the like described herein) may be inserted for the purpose of loading the code onto the computer.

In accordance with various embodiments of the present invention, a computer may run one or more software modules designed to perform one or more of the above-described operations. Such modules can, for example, be programmed using Python, Java, JavaScript, Swift, C, C++, C#, and/or another language. Corresponding program code can be placed on media such as, for example, DVD, CD-ROM, memory card, and/or floppy disk. It is noted that any indicated division of operations among particular software modules is for purposes of illustration, and that alternate divisions of operation may be employed. Accordingly, any operations indicated as being performed by one software module can instead be performed by a plurality of software modules. Similarly, any operations indicated as being performed by a plurality of modules can instead be performed by a single module. It is noted that operations indicated as being performed by a particular computer can instead be performed by a plurality of computers. It is further noted that, in various embodiments, peer-to-peer and/or grid computing techniques may be employed. It is additionally noted that, in various embodiments, remote communication among software modules may occur. Such remote communication can, for example, involve JavaScript Object Notation-Remote Procedure Call (JSON-RPC), Simple Object Access Protocol (SOAP), Java Messaging Service (JMS), Remote Method Invocation (RMI), Remote Procedure Call (RPC), sockets, and/or pipes.

Moreover, in various embodiments the functionality discussed herein can be implemented using special-purpose circuitry, such as via one or more integrated circuits, Application Specific Integrated Circuits (ASICs), or Field Programmable Gate Arrays (FPGAs). A Hardware Description Language (HDL) can, in various embodiments, be employed in instantiating the functionality discussed herein. Such an HDL can, as just some examples, be Verilog or Very High-Speed Integrated Circuit Hardware Description Language (VHDL). More generally, various embodiments can be implemented using hardwired circuitry without or without software instructions. As such, the functionality discussed herein is limited neither to any specific combination of hardware circuitry and software, nor to any particular source for the instructions executed by the data processing system.

What is claimed is:

1. A method of regenerating a FASTQ file from a compressed sequence alignment map file, the compressed sequence alignment map file including alignment data for a plurality of sequence strings for more than one aligned reads of clusters on a flow cell, the method comprising:
(a) determining an fqsum of an original FASTQ file on which the compressed sequence alignment file is based, wherein each entry of the original FASTQ file is defined by at least four line-separated fields per sequence and the fqsum represents an order invariant checksum of the original FASTQ file;
(b) storing a quality score of each base call for individual instances of the plurality of sequence strings;
(c) sorting the compressed sequence alignment map file by one or more reference genome coordinates;
(d) extracting primary alignments from the compressed sequence alignment map file;
(e) ensuring all FASTQ records from the original FASTQ file are stored in the compressed SAM file;
(f) reconstructing original base quality scores by at least one of retrieving the stored quality score or applying an inverse of a model of the stored quality score;
(g) writing at least three of the four line-separated fields per sequence of the compressed sequence alignment map file to a regenerated FASTQ file; and
(h) compressing the regenerated FASTQ file.

2. The method of claim 1, further comprising comparing the determined fqsum with an fqsum of the regenerated FASTQ file.

3. The method of claim 1, wherein said determining the fqsum of the original FASTQ file further comprises: for a set A and a set B of the original FASTQ file, fqsum ({A})=fqsum (fqsum ({A})), fqsum ({A, B})=fqsum ({B, A}), and fqsum({fqsum ({A, B}), C})=fqsum ({A, fqsum({B, C})}).

4. The method of claim 1, wherein said storing the quality score comprises generating a base quality score recalibration model.

5. The method of claim 1, wherein said storing the quality score of each base call for individual instances of the plurality of sequence strings comprises storing a Phred scaled score.

6. The method of claim 5, wherein said storing the quality score of each base call for individual instances of the plurality of sequence strings comprises storing a string of integers, equal in length to a selected sequence string of the plurality of sequence strings.

7. The method of claim 1, wherein said ensuring all FASTQ records from the original FASTQ file are stored in the compressed SAM file further comprises: (i) removing sequencing duplicates and off-target reads, and (ii) storing the sequencing duplicates and off-target reads in an auxiliary compressed sequence alignment map file.

8. The method of claim 1, wherein the at least four line-separated fields per sequence of each entry of the original FASTQ file comprises: (i) a sequence identifier field, (ii) a sequence field, (iii) a quality score identifier field, and (iv) a quality score field.

9. The method of claim 1, wherein the compressed sequence alignment map file comprises at least one of a binary alignment map file and a compressed reference-oriented map file.

10. The method of claim 1, wherein a file size of the compressed sequence alignment map file is smaller than a file size of either of the original FASTQ file and the regenerated FASTQ file.

11. A method of regenerating a FASTQ file from a compressed sequence alignment map file, the compressed sequence alignment map file including alignment data for a plurality of sequence strings for more than one aligned reads of clusters on a flow cell, the method comprising:
(a) determining an fqsum of an original FASTQ file on which the compressed sequence alignment file is based, wherein each entry of the original FASTQ file is defined by at least four line-separated fields per sequence and the fqsum represents an order invariant checksum of the original FASTQ file;
(b) storing a quality score of each base call for individual instances of the plurality of sequence strings;
(c) sorting the compressed sequence alignment map file by sequence string;
(d) extracting primary alignments from the compressed sequence alignment map file;
(e) ensuring all FASTQ records from the original FASTQ file are stored in the compressed SAM file;
(f) reconstructing original base quality scores by at least one of retrieving the stored quality score or applying an inverse of a model of the stored quality score;
(g) writing at least three of the four line-separated fields per sequence of the compressed sequence alignment map file to a regenerated FASTQ file; and
(h) compressing the regenerated FASTQ file.

12. The method of claim 11, further comprising comparing the determined fqsum with an fqsum of the regenerated FASTQ file.

13. The method of claim 11, wherein said determining the fqsum of the original FASTQ file further comprises: for a set A and a set B of the original FASTQ file, fqsum ({A})=fqsum (fqsum ({A})), fqsum ({A, B})=fqsum ({B, A}), and fqsum({fqsum ({A, B}), C})=fqsum ({A, fqsum({B, C})}).

14. The method of claim 11, wherein said storing the quality score comprises generating a base quality score recalibration model.

15. The method of claim 11, wherein said storing the quality score of each base call for individual instances of the plurality of sequence strings comprises storing a Phred scaled score.

16. The method of claim 15, wherein said storing the quality score of each base call for individual instances of the plurality of sequence strings comprises storing a string of integers, equal in length to a selected sequence string of the plurality of sequence strings.

17. The method of claim 11, wherein the at least four line-separated fields per sequence of each entry of the original FASTQ file comprises: (i) a sequence identifier field, (ii) a sequence field, (iii) a quality score identifier field, and (iv) a quality score field.

18. The method of claim 10, wherein the compressed sequence alignment map file comprises at least one of a binary alignment map file and a compressed reference-oriented map file.

19. The method of claim 11, wherein a file size of the compressed sequence alignment map file is smaller than a file size of either of the original FASTQ file and the regenerated FASTQ file.

20. A computer program product for regenerating a FASTQ file from a compressed sequence alignment map file, the compressed sequence alignment map file including alignment data for a plurality of sequence strings for more than one aligned reads of clusters on a flow cell, the computer program product being encoded on one or more machine-readable storage media and comprising instructions for:
(i) determining an fqsum of an original FASTQ file on which the compressed sequence alignment file is based, wherein each entry of the original FASTQ file is defined by at least four line-separated fields per sequence and the fqsum represents an order invariant checksum of the original FASTQ file;
(ii) storing a quality score of each base call for individual instances of the plurality of sequence strings;
(iii) sorting the compressed sequence alignment map file by one or more reference genome coordinates;
(iv) extracting primary alignments from the compressed sequence alignment map file;
(v) ensuring all FASTQ records from the original FASTQ file are stored in the compressed SAM file;
(vi) reconstructing original base quality scores by at least one of retrieving the stored quality score or applying an inverse of a model of the stored quality score;
(vii) writing at least three of the four line-separated fields per sequence of the compressed sequence alignment map file to a regenerated FASTQ file; and
(viii) compressing the regenerated FASTQ file.
